(19) United States
(12) Patent Application Publication
(10) Pub. No.: US 2018/0363054 A1
(43) Pub. Date: Dec. 20, 2018

(54) METHOD FOR FORENSIC ANALYSIS OF SEXUAL ASSAULT

(71) Applicant: University of Notre Dame du Lac, South Bend, IN (US)

(72) Inventors: Sarah LUM, South Bend, IN (US); Norman DOVICHI, South Bend, IN (US); Bonnie JASKOWSKI HUGE, South Bend, IN (US); Carlos Gusti GARTNER, South Bend, IN (US)

(73) Assignee: University of Notre Dame du Lac, South Bend, IN (US)

(21) Appl. No.: 16/013,644

(22) Filed: Jun. 20, 2018

Related U.S. Application Data
(60) Provisional application No. 62/522,496, filed on Jun. 20, 2017.

(51) Int. Cl. C12Q 1/6881; C12N 15/10; G01N 27/447; G01N 15/14

(52) U.S. Cl. CPC C12Q 1/6881; C12N 15/101; G01N 27/44791; G01N 2015/1486; G01N 27/44721; G01N 15/1484; G01N 27/44743

(57) ABSTRACT

Current procedures for the analysis of sexual assault kits are labor intensive, time consuming and deliver a success rate lower than 40%. This has resulted in rape kit backlogs of thousands. The primary challenge crime laboratories face in analyzing these cases is the separation of purified male DNA from the mixture of primarily female DNA from gynecological swabs. Effective elution of the sample from the swab and efficient separation of intact sperm cells from epithelial and other cellular debris, allow for a successful PCR amplification and short tandem repeat (STR) DNA analysis for perpetrator identification. The disclosure provides an effective and economically accessible technology for the separation of male and female cells and DNA, for example, from a gynecological swab by capillary zone electrophoresis (CZE).

### RELATED APPLICATIONS

This application claims priority under 35 U.S.C. § 119(e) to U.S. Provisional Patent Application No. 62/522,496, filed Jun. 20, 2017, which is incorporated herein by reference.

### BACKGROUND OF THE INVENTION

In the United States, sexual assaults occur on average once every two minutes and an estimated one in five women have been a victim of sexual assault. These cases have created a backlog of untested rape kits between tens of thousands to half a million. With the limitations of present technologies and policies, these numbers will undoubtedly continue to worsen. The national backlog in sexual assault case processing is due in part to the lack of an effective technology for rape kit analysis, as well as resistance to the implementation of improved methodologies.

Standard rape kits are comprised of sterile containers, bags, combs and swabs to collect and package any specimen potentially containing perpetrator DNA. Examiners collect biological fluids by swabbing the victim's genitals, rectum, mouth and body surfaces. The swabs are dried, sealed, and sent to crime labs for analysis where they are tested for the presence of sperm cells, which contain male DNA. Current methods of analysis exploit the different stabilities of the male sperm cells and female epithelial cells during extraction. A mild cell lysis step is used to recover the epithelial cell fraction from a collected swab prior to a stronger lysis step to access the sperm DNA. The swab is incubated in these solutions for a few hours or overnight. A number of centrifugation, washes, and transfers are incorporated as the sample is purified. The method is both time and labor intensive and offers, at best, a 30-40% chance of successful identification of the male perpetrator. Nevertheless, differential extraction is the most widely practiced method of sample processing in the US criminal justice system.

While the use of automation and hiring of more analysts aids in processing DNA casework, a steady increase continues in the overall backlog of rape kits. The National Institute of Justice defines a backlogged kit as, “one that has not been closed by a final report within 30 days after receipt of the evidence in the laboratory.” The average turnaround time for violent crimes including sexual assaults in the USA is 106 days.

There are a number of challenges in the use of current technologies. Differential extraction requires lengthy, often overnight incubations for optimal recovery of DNA, and consumes the entire sample in order to acquire enough DNA for amplification and analysis. Most laboratories cut the swab in two to four pieces to preserve a portion in case the initial analysis is unsuccessful. However, this practice necessitates operating on only a fraction of the available evidence. In many cases, female epithelial cells significantly outnumber the male sperm cells present on the swab, and it is very difficult to obtain a purified aliquot with enough male DNA to produce a clean short tandem repeat (STR) profile. Furthermore, the current method requires the transfer of sample to different tubes for separation, collection and analysis, and each step introduces the risk for sample contamination or loss.

In addressing the national backlog of sexual assault cases, two primary strategies have been proposed. The first is to improve key aspects of the current validated method of differential extraction. Enhanced detergents, enzymes and buffer solutions have been employed to improve the male DNA yield in standard differential extraction, though differential extraction is unlikely to achieve perfect separation. One study compared a series of buffer compositions with the standard protocol utilizing Proteinase K and an anionic detergent to remove the epithelial fraction followed by resuspension in dithiothreitol (DTT) buffer to release DNA from the sperm heads. Lounsbury et. al. (Forensic Science International: Genetics 2014, 84-89) tested citrate buffers, cellulose solution, and various detergent solutions at 42° C. and incubations for up to 24 hours. Results showed a twofold enhancement of sperm cell recovery using an anionic surfactant compared to conventional buffers from vaginal swabs with manually added semen (containing 20,000 sperm). A follow-up study performed by Lounsbury et. al. showed that a buffer containing sodium dodecyl sulfate and Proteinase K that provides nearly 90% sperm cell recovery after a half hour incubation. These studies have successfully approached the limit of effectiveness inherent in differential extraction. However, the method itself still relies on time consuming incubation steps and multiple sample transfers for extraction, centrifugation, resuspension and collection prior to analysis. These transfers are sources of contamination and sample loss. In addition, the method requires the consumption of the entire sample for a single analysis. One study found that >90% of male DNA initially present on simulated sexual assault samples was lost after standard differential DNA separation techniques.

Novel approaches such as laser capture microdissection (LCM) possess clear advantages over differential extraction by bypassing its inherent limitations. Laser capture microdissection couples a light microscope with a pulsed laser to target specific regions of sample to be extracted for DNA analysis. In one study, post-coital samples from cotton and nylon flocked swabs were deposited on slides for isolation (Budimlij a et al, Forensic Science International: Genetics 2010, 115-121). Using a robomover, sperm heads were collected and catapulted into the cap of a microfuge tube for DNA analysis. This method is specific and requires a small sample size.

However, the pulsed laser system and trained technicians required for LCM are not affordable by the majority of crime laboratories. This high overhead cost and the time it takes to process a single sample diminishes the potential of LCM systems to significantly impact the backlog in sexual assault kits or keep up with the number of new cases.

Other alternative approaches to differential extraction have been developed such as microfluidic devices or pressure cycling technologies. These strategies have shown promise in improving DNA yields compared with standard methods; however, they cannot produce a complete isolation of spermatozoa from mixture components. Furthermore, these methods rely on technology foreign to crime labs, which will make their adoption into the system more lengthy and challenging.

The development of an alternative system must overcome the limitations of current methodologies, including long separation time, separation inefficiency, inability to perform multiple sample analyses, and excess analyst interaction with sample from collection to STR profiling. Accordingly, the ideal solution would couple the leading differential extraction buffers with the specificity advantages of single cell separations. This would provide a cost effective and efficient method to separate different cell types from a mixture and to allow for rapid and accurate determination of DNA of each uncontaminated cell type in the mixture to reduce analysis backlogs in forensic laboratories.

### SUMMARY

This disclosure provides a method for capillary electrophoresis separation of an extracted mixture from a forensic sample followed by fraction collection in a system based on familiar technology currently available in US crime labs. In this work, a one-step buffer solution has been developed that elutes the sample from a gynecological swab without risking spermatozoa lysis. This initial elution step may have a major impact on sperm recovery and downstream perpetrator identification. When extraction detergents are too mild or not incubated long enough, incomplete lysis of epithelial cells leads to a contaminated DNA profile. When detergents are too harsh, or the sample remains in solution for too long, the limited number of sperm cells present may be destroyed. Studies have shown that this challenge becomes a more important issue in stored samples where spermatozoa membranes are compromised with age. An eluting solution has been developed that does not require incubation and does not have significant negative effects on stored spermatozoa over time.

Following a simple, yet effective sample elution from the swab, capillary zone electrophoresis (CZE) was used to separate intact spermatozoa from residual epithelial cells. CZE requires a very small sample size, so that the sample can be preserved for replicate analyses. This technology also provides significantly decreased sample preparation time with improved efficiency as compared to standard methods. Coupled with an automated fraction collector, the injected sample was fractionated into wells of a standard 96 well plate, and the separation was visually characterized using light microscopy prior to sperm lysis.

Accordingly, this disclosure provides a method for forensic analysis comprising:
a) mixing a buffer with a sample comprising sperm cells and epithelial cells;
b) separating sperm cells and epithelial cells in a capillary by capillary electrophoresis (CE);
c) collecting the sperm cells in a sample collector;
d) determining the concentration of the sperm cells in the sample collector; and
e) amplifying the DNA from the sperm cells by a polymerase chain reaction (PCR); wherein the sample is forensically analyzed for DNA from sperm cells.

In another embodiment, the buffer comprises tris(hydroxymethyl)aminomethane (TRIS). Additionally, this disclosure provides a method for forensic analysis of DNA comprising:
a) mixing a buffer with a sample comprising sperm and epithelial cells;
b) separating sperm and epithelial cells in a capillary by capillary isoelectric focusing;
c) collecting the sperm and epithelial cells in a sample collector;
d) determining the concentration of the sperm and epithelial cells in the sample collector; and
e) amplifying the DNA from the sperm and epithelial cells by a polymerase chain reaction (PCR);

wherein the sample is forensically analyzed by STR for DNA from the collected sperm and epithelial cells.

This disclosure also provides a buffer composition for forensic analysis comprising about 10 mM tris(hydroxymethyl)-aminomethane hydrochloride at about pH 7.5.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1. A. Instrumental design of CZE coupled with light scatter detection. Sample is placed into multipurpose block and electrokinetically injected and separated on a 60 cm, 100 μm inner diameter/160 μm outer diameter bare capillary. Light scatter is performed with a 25 mW 532 nm diode-pumped, solid state laser and detected with a single-photon counting avalanche photodiode. B. Enlarged view of cuvette.

FIG. 2. Schematic of CZE coupled with an automated fraction collector instrument. The distal end of the capillary is threaded through a Tee and aligned with the tip of the nozzle. Buffer flow is controlled at the dispensing valve and washes the fraction exiting the capillary into a plate well. The microtiter plate is fixed to a motorized microscope X-Y stage (Diagram: Huge et al., Talanta, 2014, 288-293).

FIG. 3. A. Schematic of hemacytometer. Cells in highlighted areas or touching the top or right borders of these areas are counted. The average number of cells in one region is calculated from the number multiplied by the dilution factor and divided by the volume of a single region (10⁻⁴ mL) to find the concentration of cells per milliliter of solution. B.

Demonstrated recovery of intact spermatozoa in indicated regions on a hemacytometer. Sample collected after a 5 kV/2 sec injection of unfiltered semen, background electrolyte was TRIS-HCl, pH 7.4. Separation voltage was 14 kV across a 60cm long, 100 μm inner diameter/160 μm outer diameter borosilicate capillary. CZE migration time was under 15 minutes.

FIG. 4. (a) Epithelial cells eluted from cotton swab, injected & separated on 60 cm capillary. (b) Upper trace is whole semen sample; lower trace is semen sample passed through a 0.20 μm filter to eliminate sperm cells. (c) Simulated sexual assault sample. (d) Background with 10 mM TRIS-HCl. Data analyzed using MatLab with a five-point median filter.

Example of electropherograms generated using light scatter detection. Electropherograms generate a peak at ˜10 minutes of (e) Epithelial and spermatozoa whole cell mixtures. (f) Diminished peak intensity observed when cell mixture undergoes mechanical stress through vortexing. (g) Vortexed spermatozoa lose their tails and display a cleaner migration band. (h) Background of cotton swab with 1× PBS elution; background appears after vortexed sperm. (i) Vortexed epithelial cells are mechanically lysed. (j) 10 mM TRIS-HCl pH 7.4 background electrolyte.

FIG. 5. Triplicate experiment of separation of simulated sexual assault sample eluted from a cotton swab in 10 mM TRIS-HCl. Separation performed at 233 V/cm on a 60 cm bare fused silica capillary.

FIG. 6. Fraction collection with hemacytometry quantification of intact sperm cells in a microtiter plate. Fractions were collected every ˜20 sec. An aliquot of 90 μL Trypan Blue was added to each well then magnified and counted at 500×. Highlighted wells indicate presence of intact spermatozoa. Sperm were seen to migrate in only one well at the 6 min marker. The presence of sperm in this well was verified with the EVE Automated Cell Counter.

FIG. 7. Fraction collection with EVE Automated Cell Counter quantification of intact sperm cells in a microtiter plate. Highlighted wells indicate presence of intact spermatozoa. Sperm were seen to migrate within 1 min of electropherogram peak. The EVE system also indicated small particles between 3-7 μm in size around 3 min, 8 min and 9 min. A hemacytometry analysis of these wells at 500× magnification suggested these are debris particles and not spermatozoa.

FIG. 8. Nondestructive method showing sample collection of sperm and epithelial cell mixture (a), sample extraction and elution with a compatible buffer, such as TRIS (b). Experimental parameters for CZE separation: 60 cm borosilicate capillary having a 100 μm inner diameter and a 160 μm outer diameter; 5 kV/2 sec electrokinetic injection; 14 kV separation at 233 V/cm.

### DETAILED DESCRIPTION

The primary challenge crime labs face in analyzing these cases is the separation of purified male DNA from the mixture of primarily female DNA from gynecological swabs. Effective elution of the sample from the swab and efficient separation of intact sperm cells from epithelial and other cellular debris allow for a successful polymerase chain reaction amplification and short tandem repeat (STR) analysis of the perpetrator DNA. Capillary electrophoresis (CE) is a promising tool to perform the cell separation and has three major advantages over alternative technologies: small amount of sample is consumed, which allows for replicate analyses of limited available evidence; rapid separation time compared to standard methods; and single cell detection and collection when interfaced with an automated fraction collector.

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14^{th} Edition, by R. J. Lewis, John Wiley & Sons, New York, N.Y., 2001.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrases “one or more” and “at least one” are readily understood by one of skill in the art, particularly when read in context of its usage. For example, the phrase can mean one, two, three, four, five, six, ten, 100, or any upper limit approximately 10, 100, or 1000 times higher than a recited lower limit.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements. When values are expressed as approximations, by use of the antecedent “about,” it will be understood that the particular value without the modifier “about” also forms a further aspect.

The terms “about” and “approximately” are used interchangeably. Both terms can refer to a variation of ±5%, ±10%, ±20%, or ±25% of the value specified. For example, “about 50” percent can in some embodiments carry a variation from 45 to 55 percent, or as otherwise defined by a particular claim. For integer ranges, the term “about” can include one or two integers greater than and/or less than a recited integer at each end of the range. Unless indicated otherwise herein, the terms “about” and “approximately” are intended to include values, e.g., weight percentages, proximate to the recited range that are equivalent in terms of the functionality of the individual ingredient, composition, or embodiment. The terms “about” and “approximately” can also modify the end-points of a recited range as discussed above in this paragraph.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. It is therefore understood that each unit between two particular units are also disclosed. For example, if 10 to 15 is disclosed, then 11, 12, 13, and 14 are also disclosed, individually, and as part of a range. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents. It will be further understood that the endpoints of each of the ranges are significant both in relation to the other endpoint, and independently of the other endpoint.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

The term “substantially” as used herein, is a broad term and is used in its ordinary sense, including, without limitation, being largely but not necessarily wholly that which is specified. For example, the term could refer to a numerical value that may not be 100% the full numerical value. The full numerical value may be less by about1%, about 2%, about 3%, about 4%, about 5%, about 6%, about 7%, about 8%, about 9%, about 10%, about 15%, or about 20%.

The term “Polymerase chain reaction” (PCR) refers to a technique used in molecular biology to amplify a single copy or a few copies of a segment of DNA across several orders of magnitude, generating thousands to millions of copies of a particular DNA sequence. It is an easy, cheap, and reliable way to repeatedly replicate a focused segment of DNA, a concept which is applicable to numerous fields in modern biology and related sciences.

The term “Short Tandem Repeat” (STR) analysis refers to a method in molecular biology which is used to compare specific loci on deoxyribonucleic acid (DNA) from two or more samples. A short tandem repeat is a microsatellite, consisting of a unit of two to thirteen nucleotides repeated hundreds of times in a row on the DNA strand. STR analysis measures the exact number of repeating units. STR analysis is a tool in forensic analysis that evaluates specific STR regions found on nuclear DNA. The variable (polymorphic) nature of the STR regions that are analyzed for forensic testing intensifies the discrimination between one DNA profile and another. Forensic science takes advantage of the population's variability in STR lengths, enabling scientists to distinguish one DNA sample from another. The system of DNA profiling used today is based on PCR and uses simple sequences or short tandem repeats.

The term “cell type” is a classification used to distinguish between morphologically or phenotypically distinct cell forms within a species. A multicellular organism may contain a number of widely differing and specialized cell types, such as muscle cells and skin cells in humans, that differ both in appearance and function yet are genetically identical. Cells are able to be of the same genotype, but different cell type due to the differential regulation of the genes they contain.

The term “Capillary Electrophoresis” (CE) refers to electrokinetic separation methods performed in submillimeter diameter capillaries and in micro- and nanofluidic channels. Capillary electrophoresis can be capillary zone electrophoresis (CZE) and other electrophoretic techniques. Capillary gel electrophoresis (CGE), capillary isoelectric focusing (CIEF), capillary isotachophoresis and micellar electrokinetic chromatography (MEKC) belong also to the CE class of methods. In CE methods, analytes migrate through electrolyte solutions under the influence of an electric field. Analytes can be separated according to ionic mobility and/or partitioning into an alternate phase via non-covalent interactions. Additionally, analytes may be concentrated or “focused” by means of gradients in conductivity and pH.

### Embodiments of the Invention

This disclosure provides various embodiments of a method for forensic analysis comprising:
a) mixing a buffer with a sample comprising sperm cells and epithelial cells;
b) separating sperm cells and epithelial cells in a capillary by capillary electrophoresis (CE);
c) collecting the sperm cells in a sample collector;
d) determining the concentration of the sperm cells in the sample collector; and
e) amplifying the DNA from the sperm cells by a polymerase chain reaction (PCR);

wherein the buffer comprises tris(hydroxymethyl)aminomethane (TRIS), and the sample is forensically analyzed for DNA from sperm cells.

In some embodiments, the sample is forensically analyzed via short tandem repeat (STR) for DNA. In some other embodiments, the sample collector collects eluted sperm cells, eluted epithelial cells (male, female, or both), or a combination thereof.

In additional embodiments (methods or compositions), the buffer comprises about 10 mM TRIS at about pH 7.5. In other embodiments the concentration of TRIS is about 1 mM to about 100 mM. In other embodiments, the pH of the buffer is about 6.9 to about 8.1. In further embodiments, the buffer further comprises about 1% sodium dodecyl sulfate (SDS). In yet other embodiments the percent of SDS is about 0.5% to about 10%. In yet additional embodiments, the buffer comprises a phosphate buffered saline (PBS), for example, 10 mM phosphate buffer, 2.7 mM potassium chloride, 137 mM sodium chloride, and 1.76 mM potassium phosphate.

In various embodiments, the capillary has an inner diameter about the diameter of an epithelial cell. In other embodiments, the epithelial cells are human female or male epithelial cells. In further embodiments, the capillary has an inner diameter larger than a single cell, or about 50% to about 150% larger than a single cell, or inner diameter of about 80 μm to about 120 μm. In various embodiments, the capillary has an inner diameter about the diameter of an epithelial cell or up to about five times the diameter of an epithelial cell. In some other embodiments, the inner diameter is about 50 microns, about 60 microns, about 70 microns, about 80 microns, about 90 microns, about 100 microns, about 20 microns to about 200 microns, or about 60 microns to about 600 microns. The inner diameter can also be about 2 times, about 3 times, about 4 times, about 6 times, or about 8 times the diameter of an epithelial cell or a sperm cell. In other embodiments, the capillary has an outer diameter of at least 100 μm.

In yet other embodiments, the capillary has a length of about 30 cm to about 100 cm, and has an inner diameter of about 40 microns to about 120 microns. In some embodiments the length is at least 30 cm, or the length is about 40 cm, about 50 cm, about 60 cm, about 70 cm, or about 80 cm.

In additional embodiments, the concentration of sperm cells is determined with a hemacytometer. In yet other embodiments, the sample is electrokinetically injected into the capillary at about 1 kV to about 10 kV. Injection can be at about 2 kV, about 4 kV, about 6 kV, or about 8 kV, or at about 0.5 kV to about 20 kV. In other embodiments, the sample is electrokinetically injected into the capillary for about 0.1 seconds to about 30 seconds. The length of injection can be also about 1 sec, about 2 sec, about 3 sec, or about 4 sec, or about 0.1 seconds to about 30 seconds.

In various embodiments, the sample is separated in the capillary at a potential of about 5 kV to about 25 kV. In other embodiments, the separation is at about 10 kV, about 15 kV, or about 20 kV, or about 5 kV to about 50 kV. In additional embodiments, the sample is separated in the capillary at an electric field of about 100 V/cm to about 500 V/cm. In additional embodiments, the resolution is about 150 V/cm, about 200 V/cm, about 250 V/cm, about 300 V/cm, about 350 V/cm, about 400 V/cm, or about 450 V/cm.

In additional embodiments, the method comprises the step of detecting the sperm cells eluting from the capillary wherein the sperm cells are detected by light scattering or fluorescence. In yet other embodiments, the sperm cells are detected by light scattering of laser light, wherein the wavelength of the laser light is about 532 nm. In other embodiments the wavelength is about 400 nm to about 600 nm. In further embodiments, the cells have a migration time determined by a light scattering detector.

Various other embodiments further comprise a short tandem repeat (STR) analysis of the collected cells. In other embodiments, the collected cells are sperm cells, epithelial cells or a combination thereof.

In additional embodiments, the capillary is a silica capillary, and wherein sperm cells and epithelial cells are separated in less than about 60 minutes. In some embodiments, the capillary is a borosilicate capillary. In other embodiments, the interior surface of the capillary is coated or uncoated. In some embodiments, separation is in less than about 45 minutes, 30 minutes, or 15 minutes. In yet other embodiments, the sperm cells are collected by an automated fraction collector. In further embodiments, the fraction collector is fitted with a 96-well plate, a 384-well plate, or 1 mL or smaller collection tubes.

In some embodiments, the sample is a gynecological swab or a buccal swab. In further embodiments, the sample is from a gynecological swab, a buccal swab, a condom, bedding, or clothing. The sample can also be from any type of material, such as fabrics, underwear, surfaces, etc., that was soiled by cells, bodily fluids, or genetic material. In yet some other embodiments the sample is any type of forensic sample. In various additional embodiments, the forensic analysis provides evidence of sexual assault. In other embodiments, the male DNA and female DNA are separated.

This disclosure also provides a buffer composition for forensic analysis comprising about 10 mM tris(hydroxymethyl)-aminomethane hydrochloride at about pH 7.5. In other embodiments, the composition comprises about 1% sodium dodecyl sulfate (SDS).

Various embodiments of this disclosure provide a method for forensic analysis of DNA comprising:
a) mixing a buffer with a sample comprising sperm and epithelial cells;
b) separating sperm and epithelial cells in a capillary by capillary isoelectric focusing (or a related technique in some other embodiments);
c) collecting the sperm and epithelial cells in a sample collector;
d) determining the concentration of the sperm and epithelial cells in the sample collector; and
e) amplifying the DNA from the sperm and epithelial cells by a polymerase chain reaction (PCR);

wherein the sample is forensically analyzed by STR for DNA from the collected sperm and epithelial cells.

This disclosure provides ranges, limits, and deviations to variables such as volume, mass, percentages, ratios, etc. It is understood by an ordinary person skilled in the art that a range, such as “number 1” to “number 2”, implies a continuous range of numbers that includes the whole numbers and fractional numbers. For example, 1 to 10 means 1, 2, 3, 4, 5, . . . 9, 10. It also means 1.0, 1.1, 1.2. 1.3, . . . , 9.8, 9.9, 10.0, and also means 1.01, 1.02, 1.03, and so on. If the variable disclosed is a number less than “number 10”, it implies a continuous range that includes whole numbers and fractional numbers less than number 10, as discussed above. Similarly, if the variable disclosed is a number greater than “number 10”, it implies a continuous range that includes whole numbers and fractional numbers greater than number 10. These ranges can be modified by the term “about”, whose meaning has been described above.

### Results and Discussion

Sample Preparation Buffers. Results shown in Table 1 indicate that TRIS and SDS were superior in eluting spermatozoa from the swab by at least a factor of two compared to PBS. The second experiment shown in Table 2 demonstrated that TRIS maintained cell viability considerably better than SDS, maintaining 14% more cells in the day 2 solution.

Evaluation of Sample Preparation Buffers. Alternative detergents, enzymes and buffers have been employed to improve the male DNA yield in standard differential extraction practices. Common components of these solutions include Proteinase K (ProK) and/or DNase to degrade epithelial cells and free DNA, an anionic detergent such as sodium dodecyl sulfate (SDS) to aid in elution of the sample from the swab, and dithiothreitol (DTT) to access sperm cell DNA. However, each of these components has been shown to degrade the spermatozoa membrane, which protects the target DNA of interest. This degradation is particularly important for aged samples because sperm cell membranes become compromised over time. Using CZE as the primary separation technique eliminated the need for preliminary separation in the swab elution procedure. In order to cut down on time consuming incubation steps and multiple sample transfers for extraction, centrifugation, resuspension and collection prior to sample analysis, a single-step sample elution buffer was designed. In selecting the ideal buffer, three primary objectives were deemed important: effective elution of the sample from the swab, preservation of spermatozoa, and compatibility with CZE separation.

Phosphate buffered saline (PBS-10 mM phosphate buffer, 2.7 mM potassium chloride, 137 mM sodium chloride, and 1.76 mM potassium phosphate) was selected because it is a common solution utilized to simulate mammalian cell environments by maintaining a physiological pH range of 6.9-7.4 and physiological ionic strength.

A solution of 10 mM TRIS-HCl was prepared because it is a common component of elution buffers including the optimized one-step buffer designed by Lounsbury et. al. (Forensic Science International: Genetics 2014, 84-89). Similar to PBS, its ionic strength and pH is ideal for long-term sample storage. TRIS is also compatible with CZE separations, maintaining a consistent current during the course of a separation, thereby giving highly reproducible migration times.

Multiple studies have suggested that solutions with a small percentage of SDS improves efficiency of sample elution because SDS acts as a detergent that releases cells from cotton swab fibers.

As expected, these results show that PBS was the optimal storage buffer for cells. A negligible amount of sperm cells lysis was observed following storage in PBS overnight. However, the results show that TRIS and 1% SDS effectively eluted ˜3x's the number of sperm cells from dried cotton swabs as did PBS. Between these two solutions, TRIS maintained cell viability significantly more effectively and was therefore selected as the extraction, separation and storage buffer.

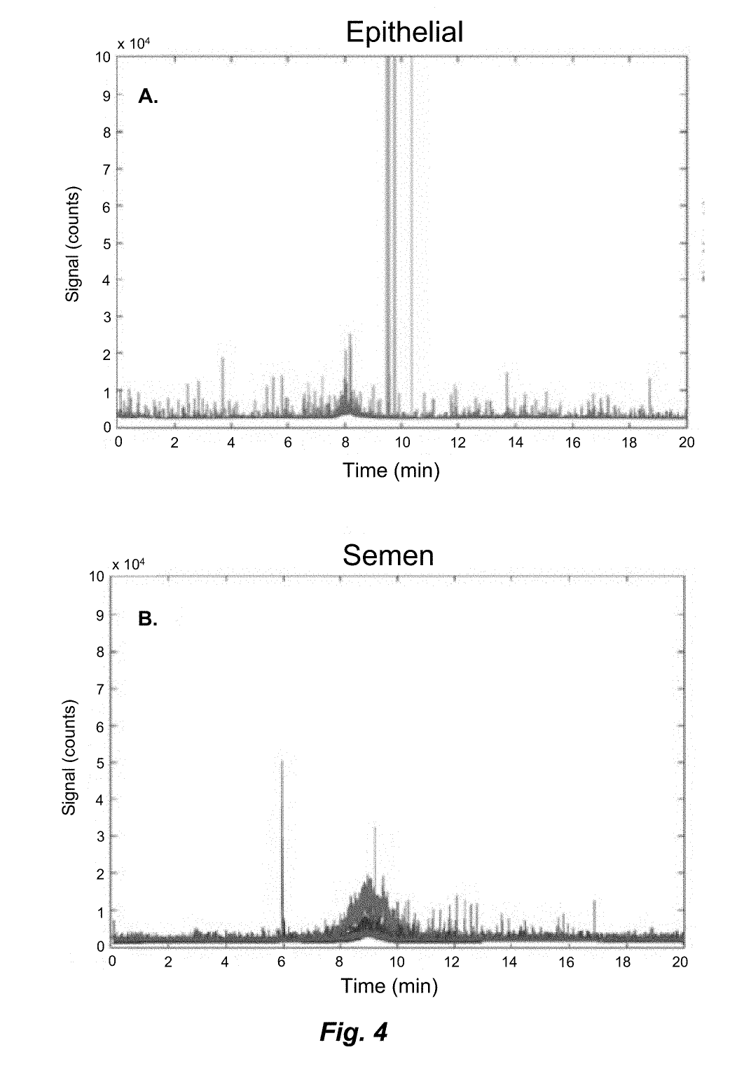

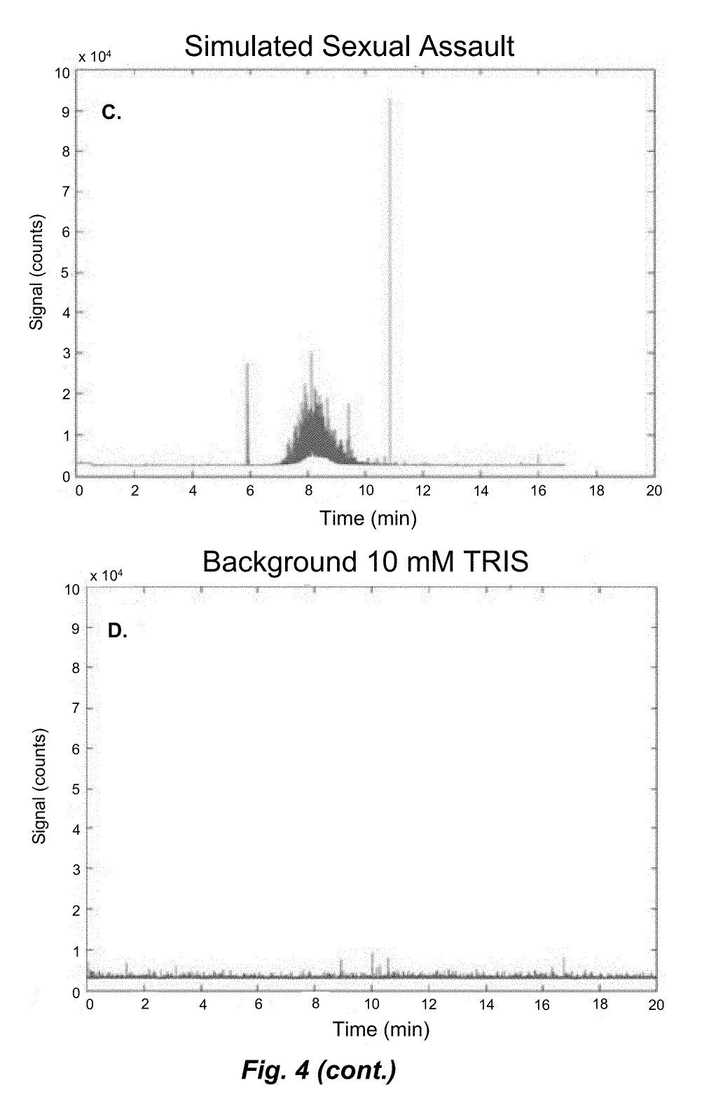

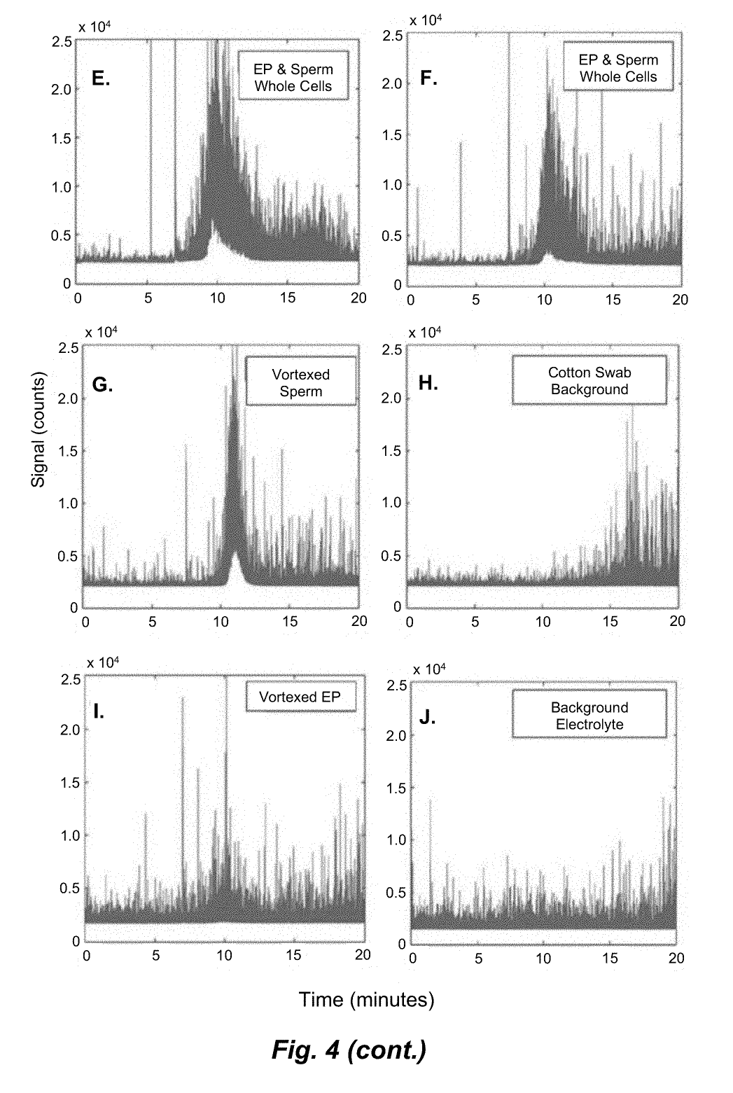

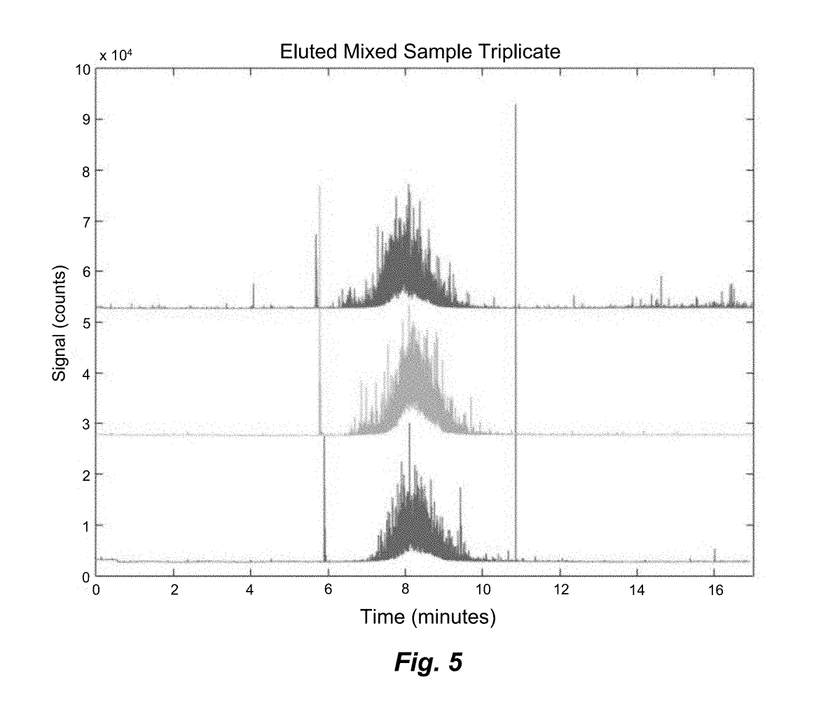

CZE Detection Methods. Results shown in FIG. 4 demonstrate the separation of sperm from epithelial cells and lysed cellular debris in under 15 mins. Data collected in Labview was processed in Matlab and treated with a five-point median filter to remove noise spikes generated from particles passing through the laser line. FIG. 5 demonstrates the reproducibility of simulated sexual assault sample migration through CE. The capillary was flushed at 10 psi for 1 min each with 1M NaOH, deionized water and 10 mM TRIS-HCl between each experiment.

Evaluation of CZE Detection Methods. A number of detection methods were considered to verify the migration time of sample through CZE separations. Laser induced fluorescence could be performed with Acridine Orange/Propidium Iodine to stain nucleated cells (both epithelial and sperm) and indicate viability. Sperm-specific antibodies were also considered to further identify sperm heads from lysed cellular debris. Lastly, the incorporation of magnetic beads coupled with antibodies has shown heightened efficiency in selectively capturing sperm cells. However, all these methods would have an impact on the electrophoretic properties of the spermatozoa by altering their charge or size and therefore be non-ideal not cell-specific, it was selected for detection because it is label-free and sensitive enough to detect 40-60 μm epithelial cells and 5 μm sperm cells.

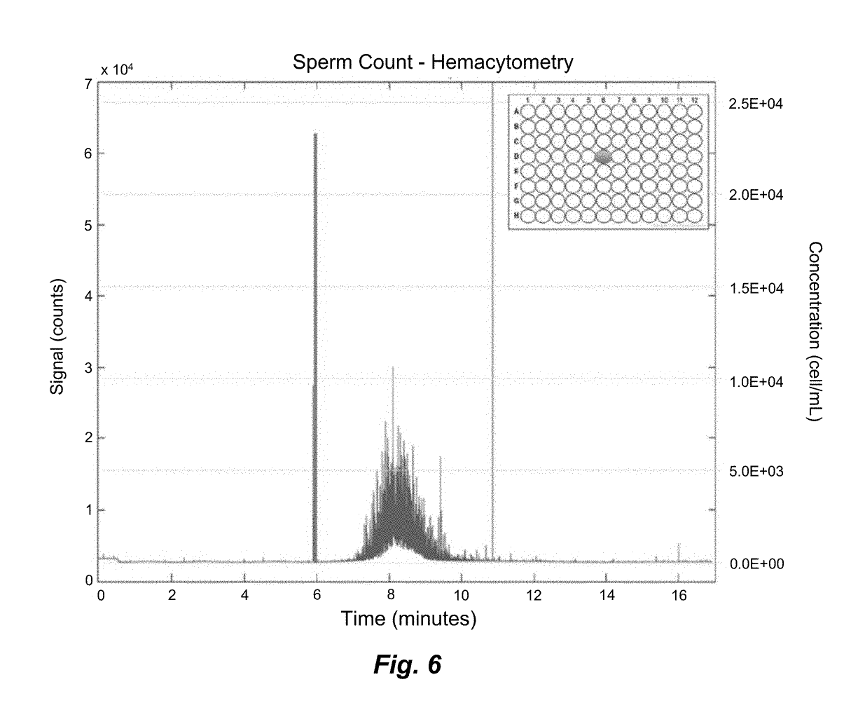

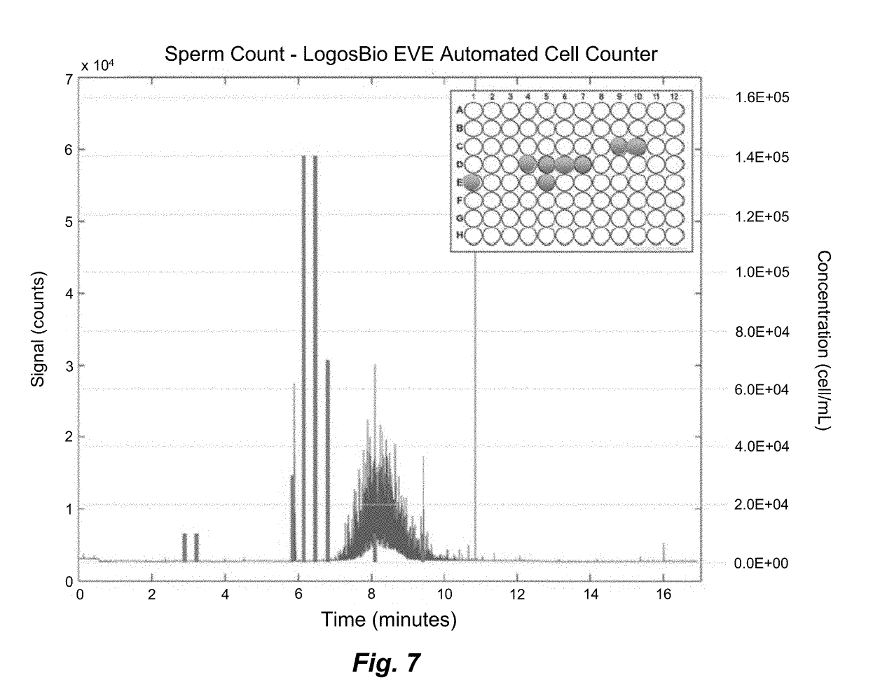

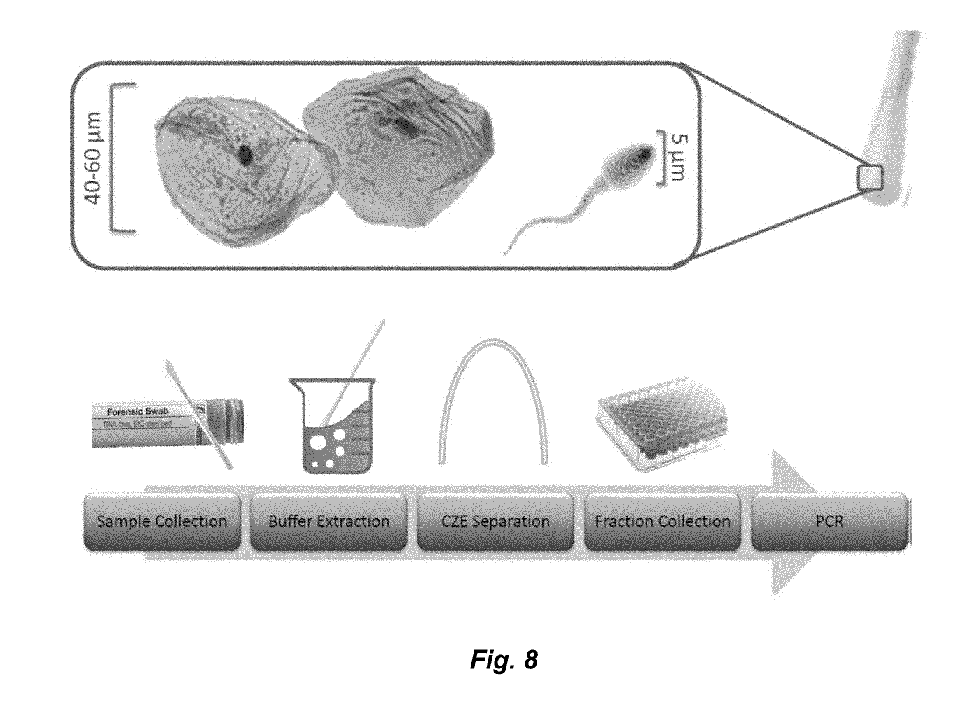

Fraction Collection on a Microtiter Plate with Analysis by Hemacytometry. Intact sperm cell collection was verified with light microscopy corresponding to CZE migration times predicted by light scatter detection. Table 3 and FIG. 6 show the sperm collection and quantification on a hemacytometer and Table 4 and FIG. 7 show quantification performed by the LogosBio EVE automated cell counting system.

Evaluation of Fraction Collection on a Microtiter Plate. Deposition of sample aliquots using an automated fraction collector on a microtiter plate provided a useful format for downstream DNA analysis of components. The standard 96 well plate fits well into the traditional crime laboratory workflow and current PCR instrumentation. Aliquots can be programmed to deposit species of similar electrophoretic mobilities (sperm, lysed cellular debris or epithelial cells) into a specific well, or multiple wells depending on preferred analysis methods. In this work, the deposition period was set to 20 seconds to accurately determine the window of spermatozoa migration. Like all human cells, spermatozoa differ slightly in size and shape, which will impact their migration time.

A reservoir of deposition buffer (10 mM TRIS-HCl) released 10 μL of solution into each well along with nanoliters of capillary eluate. This deposition buffer prevented sample loss through evaporation due to the small volume of sample that is released from the capillary. The deposition buffer also ensured no cross contamination or carryover between wells.

Evaluation of Analysis with Hemacytometry. Though there are a handful of cell-counting technologies commercially available, hemacytometry is the industry gold standard. Two different automated cell counters were evaluated. While they performed reasonably well for epithelial cells, the smaller sperm cells were at the lower end of their limit of detection (˜5 μm). The instruments were programmed to identify spherical cells with a darker outer membrane outline, a lighter inner membrane region and a nucleus. They failed to correctly identify sperm cells within the mixture likely due to their elliptical shape and entirely dark body. Some darker regions of epithelial cells or cellular debris were identified as spermatozoa while many sperm heads were glossed over. Results above include the evaluation of the LogosBio EVE Automated Cell Counter. A hemacytometry analysis of wells indicated to contain spermatozoa, revealed cellular debris of similar size to sperm. Therefore, a hemacytometer count was relied on to provide accurate information on the location of whole spermatozoa in the microtiter plate.

TABLE 3
Fractions were collected every 20 seconds beginning in well C1 and
following a serpentine pattern on the microtiter plate. Sperm count was
performed by pipetting 90 μL Trypan Blue dye into each well, then
loading 10 μL of solution onto the hemacytometer. Cells in 5 of 9 grids
on the hemacytometer were visualized at 500x magnification, counted
and averaged. The concentration was calculated by multiplying by a
dilution factor of 10 and dividing by the volume of the well, 10⁻⁴ mL.
Sperm Count - Hemacytometer

| Time (sec) | Corresponding Well | Cell Count | Concentration (cell/mL) |
|---|---|---|---|
| 0 | C1 | 0 | 0.0E+00 |
| 20 | C2 | 0 | 0.0E+00 |
| 40 | C3 | 0 | 0.0E+00 |
| 60 | C4 | 0 | 0.0E+00 |
| 80 | C5 | 0 | 0.0E+00 |
| 100 | C6 | 0 | 0.0E+00 |
| 120 | C7 | 0 | 0.0E+00 |
| 140 | C8 | 0 | 0.0E+00 |
| 160 | C9 | 0 | 0.0E+00 |
| 180 | C10 | 0 | 0.0E+00 |
| 200 | C11 | 0 | 0.0E+00 |
| 220 | C12 | 0 | 0.0E+00 |
| 240 | D12 | 0 | 0.0E+00 |
| 260 | D11 | 0 | 0.0E+00 |
| 280 | D10 | 0 | 0.0E+00 |
| 300 | D9 | 0 | 0.0E+00 |
| 320 | D8 | 0 | 0.0E+00 |
| 340 | D7 | 0 | 0.0E+00 |
| 360 | D6 | 2 | 2.0E+04 |
| 380 | D5 | 0 | 0.0E+00 |
| 400 | D4 | 0 | 0.0E+00 |
| 420 | D3 | 0 | 0.0E+00 |
| 440 | D2 | 0 | 0.0E+00 |
| 460 | D1 | 0 | 0.0E+00 |
| 480 | E1 | 0 | 0.0E+00 |
| 500 | E2 | 0 | 0.0E+00 |
| 520 | E3 | 0 | 0.0E+00 |
| 540 | E4 | 0 | 0.0E+00 |
| 560 | E5 | 0 | 0.0E+00 |
| 580 | E6 | 0 | 0.0E+00 |
| 600 | E7 | 0 | 0.0E+00 |
| 620 | E8 | 0 | 0.0E+00 |
| 640 | E9 | 0 | 0.0E+00 |
| 660 | E10 | 0 | 0.0E+00 |
| 680 | E11 | 0 | 0.0E+00 |
| 700 | E12 | 0 | 0.0E+00 |
| 720 | F12 | 0 | 0.0E+00 |
| 740 | F11 | 0 | 0.0E+00 |
| 760 | F10 | 0 | 0.0E+00 |
| 780 | F9 | 0 | 0.0E+00 |
| 800 | F8 | 0 | 0.0E+00 |
| 820 | F7 | 0 | 0.0E+00 |
| 840 | F6 | 0 | 0.0E+00 |
| 860 | F5 | 0 | 0.0E+00 |
| 880 | F4 | 0 | 0.0E+00 |
| 900 | F3 | 0 | 0.0E+00 |

TABLE 4
Sperm count was performed by pipetting 10 μL Trypan Blue
dye into each well, then loading 10 μL of solution onto the EVE slide.
The sample was visualized and focused on an LCD screen. Cell
identification parameters were set to a minimum size of 3 μm,
a maximum size of 7 μm with 30% roundness. The concentration was
calculated by multiplying by a dilution factor of 2 and dividing by the
volume of the chamber, 10⁻³ mL.
Sperm Count - LogosBio EVE Automated Cell Counter

| Time (sec) | Corresponding Well | Cell Count | Concentration (cell/mL) |
|---|---|---|---|
| 0 | C1 | 0 | 0.0E+00 |
| 20 | C2 | 0 | 0.0E+00 |
| 40 | C3 | 0 | 0.0E+00 |
| 60 | C4 | 0 | 0.0E+00 |
| 80 | C5 | 0 | 0.0E+00 |
| 100 | C6 | 0 | 0.0E+00 |
| 120 | C7 | 0 | 0.0E+00 |
| 140 | C8 | 0 | 0.0E+00 |
| 160 | C9 | 1 | 2.0E+03 |
| 180 | C10 | 1 | 2.0E+03 |
| 200 | C11 | 0 | 0.0E+00 |
| 220 | C12 | 0 | 0.0E+00 |
| 240 | D12 | 0 | 0.0E+00 |
| 260 | D11 | 0 | 0.0E+00 |
| 280 | D10 | 0 | 0.0E+00 |
| 300 | D9 | 0 | 0.0E+00 |
| 320 | D8 | 0 | 0.0E+00 |
| 340 | D7 | 3 | 6.0E+03 |
| 360 | D6 | 14 | 2.8E+04 |
| 380 | D5 | 14 | 2.8E+04 |
| 400 | D4 | 7 | 1.4E+03 |
| 420 | D3 | 0 | 0.0E+00 |
| 440 | D2 | 0 | 0.0E+00 |
| 460 | D1 | 0 | 0.0E+00 |
| 480 | E1 | 1 | 2.0E+03 |
| 500 | E2 | 0 | 0.0E+00 |
| 520 | E3 | 0 | 0.0E+00 |
| 540 | E4 | 0 | 0.0E+00 |
| 560 | E5 | 1 | 2.0E+03 |
| 580 | E6 | 0 | 0.0E+00 |

### Conclusion

In this work, a novel CZE-Fraction Collection system was developed to provide rapid separation and collection of purified, intact spermatozoa for the analysis of sexual assault kits.

Parameters for sample extraction from a gynecological swab were designed to maintain the integrity of spermatozoa and are compatible with CZE separation. Spermatozoa migrate in a narrow band in under 15 minutes, are collected in designated wells of a standard microtiter plate and the collection of intact cells post separation was verified with light microscopy. This technology may aid crime laboratories in processing new sexual assault cases as well as in eliminating the backlog of kits by drastically decreasing analysis time while providing successful perpetrator identifications. The heightened sensitivity of CZE provides the ability to isolate spermatozoa from a complex mixture without the use of harsh detergents or extraction procedures that may damage aged samples. This automated fraction collector is programmable to deposit purified aliquots into designated wells of a microtiter plate. Furthermore, this technology is compatible within the context of public and private laboratories as elution of sample from a collected swab and the standard 96 well plate output is compatible with commercial PCR systems in crime laboratories and can readily be incorporated into their standard workflow.

The following Examples are intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

### EXAMPLES

### Example 1. Sample Collection

Semen samples from healthy volunteers were collected by the Hall Lab at the University of Illinois at Chicago (UIC) and stored at −20° C. Institution Review Board approval with UIC for sample collection is on file. Semen samples were transported on dry ice to the University of Notre Dame (UND) where they were aliquoted into 500 μL portions and stored at −80° C. until ready for use.

Epithelial buccal swabs were collected from healthy volunteers at the University of Notre Dame. Samples were collected by swabbing the inside of either cheek for 15 seconds with a 6-inch cotton-tipped wooden applicator. Swabs were air dried for a minimum of 24 h in a temperature and humidity-controlled environment prior to use. Institution Review Board approval with UND for sample collection is on file.

### Example 2. Determination of Optimal Elution Buffer

A solution of 1× PBS at a pH of 7.4 was purchased from VWR and evaluated for its ability to extract sample from a cotton swab.

A solution of 10 mM Tris(hydroxymethyl)aminomethane (TRIS) was prepared with ultrapure water and filtered with a Thermo Scientific Nalgene bottle-top vacuum filter system with a 50 mm Filter Unit. The solution was titrated to a pH of 7.5 with 1 M HCl. A solution of 1% SDS was made with 10 mM TRIS-HCl (described above) and titrated to a pH of 7.5 with 1 M HCl.

A sample swab was prepared by depositing 50 μL of semen onto a cotton tipped wooden applicator and air dried at room temperature overnight. The swab was then placed in a 1.5 mL microcentrifuge tube and a 0.5 mL aliquot of extraction solution (1× PBS, TRIS-HCl or 1% SDS) was pipetted over the swab. The sample was gently vortexed for 30 seconds on setting of 1.5 on a Fisher Vortex Genie 2. The swab tip was pressed against the walls of the microcentrifuge tube to release excess liquid, then discarded. The tube containing the eluted sample was vortexed for 1 second before transferring 100 μL of cell solution into a new tube which contained 500 μL of 0.4% Trypan Blue dye and 400 μL of 1× PBS, pH 7.4, for a total volume of 1 mL. The solution was allowed to incubate for 5 min. The sample was hand mixed briefly prior to loading 10 μL of sample onto a hemacytometer for a cell count. Eluted cell samples were stored in their elution buffer at 4° C. until a second count was performed to evaluate the long-term viability of cells stored in each condition.

### Example 3. Preparation of Semen Swabs

A 1:10 dilution of semen and 10 mM TRIS-HCl buffer (pH 7.5) was prepared for deposition on autoclaved sterile cotton swabs. Swabs were inserted into a 1.5 mL microcentrifuge tube containing the diluted semen mixture and absorbed an average of 160 μL of sample. The swabs were stored at room temperature in a humidity-controlled environment for a minimum of 24 h prior to use.

### Example 4. Preparation of Simulated Sexual Assault Swabs

Simulated sexual assault samples were composed by depositing TRIS-HCl diluted semen samples onto epithelial buccal swabs in a similar manner as described above. The swab was allowed to air dry for a minimum of 24 h at room temperature prior to use.

After air drying, the sample swab was placed in a 1.5 mL microcentrifuge tube and washed with 0.5 mL of 10 mM TRIS-HCl buffer. The tube was lightly vortexed for 60 seconds. After vortexing, the swab was removed from the tube and its contents were ready for analysis by hemacytometry.

### Example 5. Light Scatter Detection

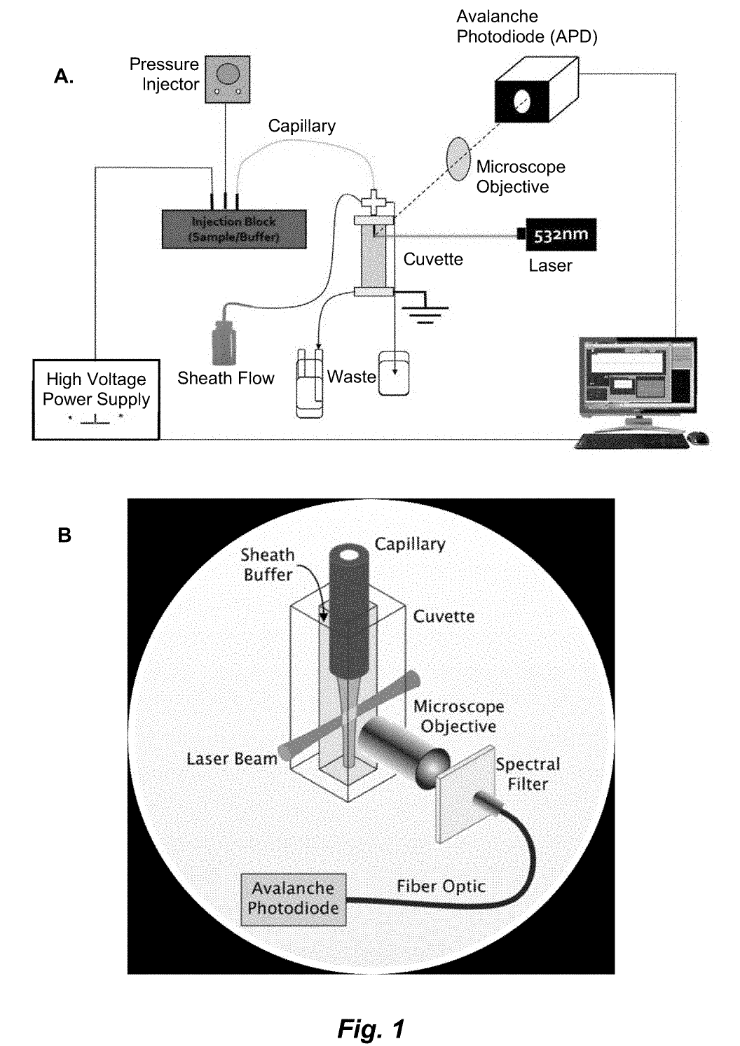

The sample was separated through a capillary electrophoresis (CE) instrument, which has been described (Dada et al, Analyst, 2012, 3099-33101) in detail elsewhere (FIG. 4). Briefly, the sample is placed in a multi-purpose injection block connected to a Spellman CZE1000R high voltage power supply and injected for 2 seconds at 5 kV. The sample is separated at 233 V/cm on a 60 cm bare silica capillary with an inner diameter of 100 μm (Polymicro). The capillary is pretreated with a 1 min flush at 10 psi of methanol, deionized water, 1 M NaOH, deionized water and 10 mM TRIS-HCl. The capillary is flushed for 1 min at 10 psi with 1 M NaOH, deionized water and 10 mM TRIS-HCl between each experiment. The sample stream was placed at the laser beam waist in the center of a sheath flow cuvette. A solution of 10 mM TRIS-HCl at a pH of 7.5 was used as the background electrolyte. Light scatter detection was achieved with a 25 mW, 532 nm diode-pumped solid-state laser beam (CrystaLaser) and collected at right angles. The migration of individual components was determined with light scatter detected by a single-photon counting avalanche photodiode. Software written in Labview (National Instruments) controlled injection and separation voltages and recorded the light scatter signal. The instrument design is shown in FIG. 1.

### Example 6. Fraction Collection

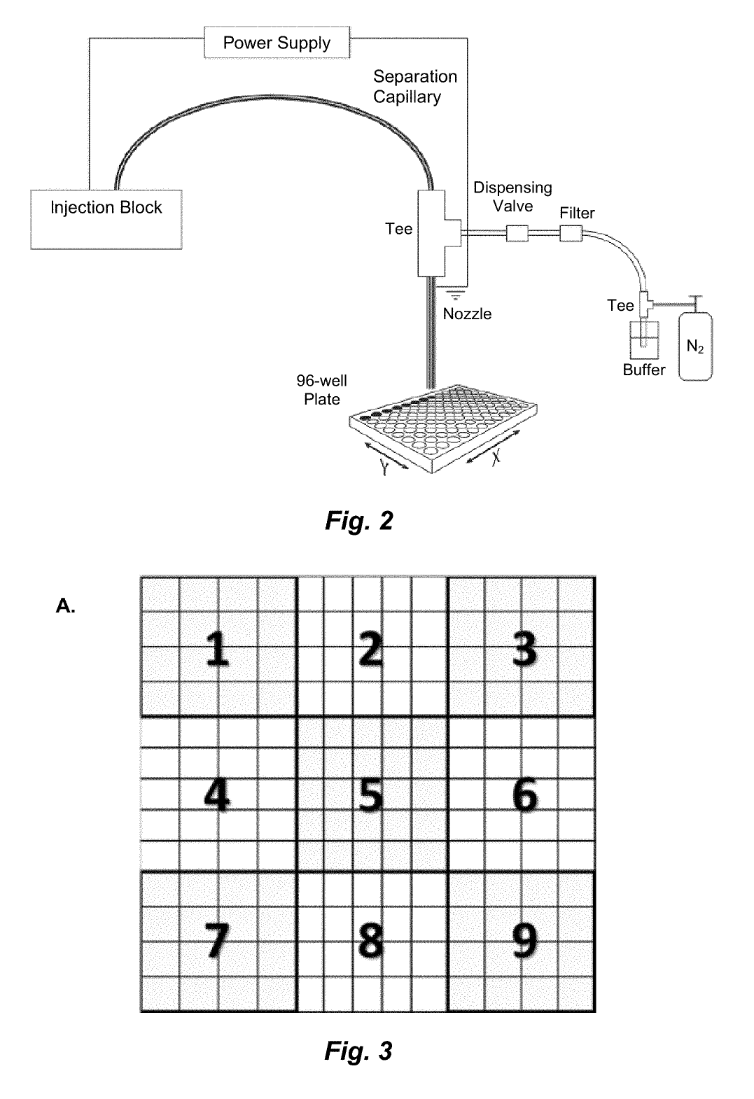

Separation parameters were replicated on a second CZE system coupled with an automated fraction collector (FIG. 2) which has been described in detail elsewhere (Huge et al, Talanta, 2014, 288-293). Briefly, the distal end of the capillary was inserted through a Tee fitting and aligned with the end of a metal nozzle secured about 2 mm above the microtiter plate. The Tee was connected to a second tube that contained deposition buffer (10 mM TRIS-HCl) maintained at 10 psi with nitrogen gas. The microtiter plate was mounted on a motorized microscope stage programmed to shift at select time points to allow sample deposition in the center of each well of a microtiter plate. In order to achieve consistent time intervals between sample deposition, fraction collection was performed in a serpentine-like fashion with the stage moving in the X direction down odd rows and the −X direction down even rows.

A software designed in Labview controlled the motion of the stage, fraction deposition period and injection and separation voltages. In this experiment, the fraction collector was programmed to deposit a ˜10 μL droplet containing deposition buffer and capillary eluate into a standard 96 well plate at 20 second intervals. The plate was stored at 4° C. until analysis.

### Example 7. Hemacytometry

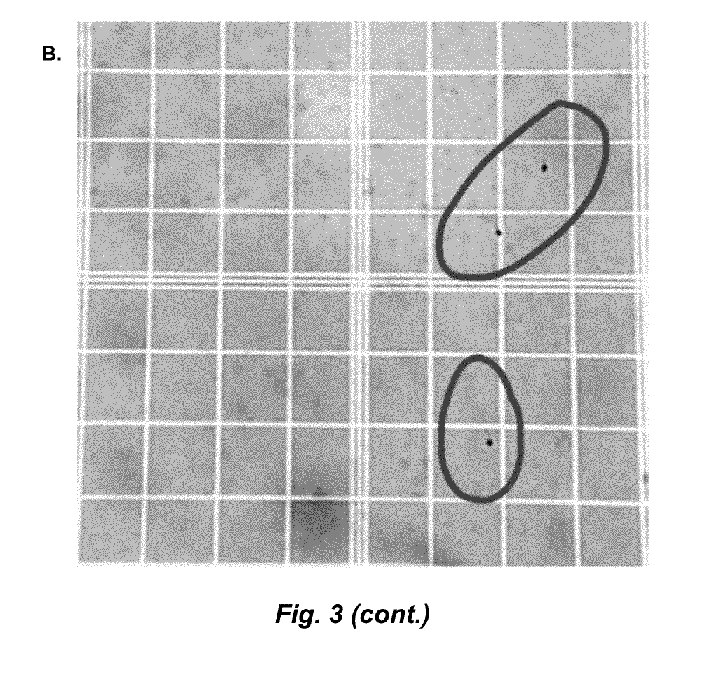

A visual analysis using light microscopy was performed to quantitatively evaluate the contents of each well on the microtiter plate. An aliquot of 90 μL 0.4% Trypan Blue was added to each well containing 10 μL of sample and allowed to incubate for 1 min. A Reichert Bright-Line hemacytometer was used to quantify the sperm and epithelial cell concentrations. A 10 μL aliquot of the cell solution was loaded onto the hemacytometer and imaged at 100×, 200× or 500× magnification. The cells in regions 1,3,5,7, and 9 (see FIG. 3) were counted. The mean of this count was multiplied the dilution factor of 10 and divided by the volume of the hemacytometer wells, 10⁻⁴ mL, to obtain the cell concentration in each well. Every count was performed in triplicate. The hemacytometer was washed with 70% EtOH and dried between uses.

### SUMMARY

Capillary zone electrophoresis (CZE) is a promising tool to perform the cell separation and has three major advantages over alternative technologies: small amount of sample is consumed, which allows for replicate analyses of limited available evidence; rapid separation time compared to standard methods; and single cell detection and collection when interfaced with an automated fraction collector developed in-house. In this work, simulated sexual assault samples are eluted from cotton swabs and the mixture is directly injected into a novel CZE system where intact cells and lysed cellular matrices are separated by their unique electrophoretic properties.

Thus, a novel CZE-Fraction Collection system was developed to provide rapid separation and collection of purified, intact spermatozoa for the analysis of sexual assault kits. Parameters for sample extraction from a gynecological swab were designed to maintain the integrity of spermatozoa and are compatible with CZE separation. Simulated sexual assault samples, prepared by mixing semen with epithelial cells generated from buccal swabs, are eluted in a mild buffer solution at physiological pH to maintain intact spermatozoa which are verified through light microscopy. The sample is injected in a capillary and separated. Results have shown that spermatozoa migrate in a confined band in less than 15 minutes. The CZE instrument was coupled with an automated fraction collector where the sample was collected into individual wells on a microtiter plate. Each well corresponds to a CZE migration time interval. Light microscopy was used to confirm the separation and collection of intact sperm cells at designated time points. The isolated sample then underwent PCR amplification and STR analysis for forensic identification.

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

All publications, patents, and patent documents are incorporated by reference herein, as though individually incorporated by reference. No limitations inconsistent with this disclosure are to be understood therefrom. The invention has been described with reference to various specific and preferred embodiments and techniques. However, it should be understood that many variations and modifications may be made while remaining within the spirit and scope of the invention.

What is claimed is:

1. A method for forensic analysis comprising:
a) mixing a buffer with a sample comprising sperm cells and epithelial cells;
b) separating sperm cells and epithelial cells in a capillary by capillary electrophoresis (CE);
c) collecting the sperm cells in a sample collector;
d) determining the concentration of the sperm cells in the sample collector; and
e) amplifying the DNA from the sperm cells by a polymerase chain reaction (PCR);
wherein the buffer comprises tris(hydroxymethyl)aminomethane (TRIS), and the sample is forensically analyzed for DNA from sperm cells.

2. The method of claim 1 wherein the buffer comprises about 10 mM TRIS at about pH 7.5.

3. The method of claim 2 wherein the buffer further comprises about 1% sodium dodecyl sulfate (SDS).

4. The method of claim 1 wherein the capillary has an inner diameter about the diameter of an epithelial cell or up to about five times the diameter of an epithelial cell.

5. The method of claim 4 wherein the epithelial cells are human female or male epithelial cells.

6. The method of claim 1 wherein the capillary has a length of about 30 cm to about 100 cm, and has an inner diameter of about 40 microns to about 120 microns.

7. The method of claim 1 wherein the concentration of sperm cells is determined with a hemacytometer.

8. The method of claim 1 wherein the sample is electrokinetically injected into the capillary at about 1 kV to about 10 kV.

9. The method of claim 1 wherein the sample is electrokinetically injected into the capillary for about 0.1 seconds to about 30 seconds.

10. The method of claim 1 wherein the sample is separated in the capillary at a potential of about 5 kV to about 25 kV.

11. The method of claim 1 wherein the sample is separated in the capillary at an electric field of about 100 V/cm to about 500 V/cm.

12. The method of claim 1 further comprising detecting the sperm cells eluting from the capillary wherein the sperm cells are detected by light scattering or fluorescence.

13. The method of claim 12 wherein the sperm cells are detected by light scattering of laser light, wherein the wavelength of the laser light is about 532 nm.

14. The method of claim 1 further comprising a short tandem repeat (STR) analysis of the sperm cells.

15. The method of claim 1 wherein the capillary is a silica capillary, and wherein sperm cells and epithelial cells are separated in less than about 60 minutes.

16. The method of claim 1 wherein the sperm cells are collected by an automated fraction collector.

17. The method of claim 1 wherein the sample is from a gynecological swab, a buccal swab, a condom, bedding, or clothing.

18. The method of claim 17 wherein the forensic analysis provides evidence of sexual assault.

19. A buffer composition for forensic analysis comprising about 10 mM tris(hydroxymethyl)-aminomethane hydrochloride at about pH 7.5.

20. The buffer composition of claim 19 further comprising about 1% sodium dodecyl sulfate (SDS).

21. A method for forensic analysis of DNA comprising:
a) mixing a buffer with a sample comprising sperm and epithelial cells;
b) separating sperm and epithelial cells in a capillary by capillary isoelectric focusing;
c) collecting the sperm and epithelial cells in a sample collector;
d) determining the concentration of the sperm and epithelial cells in the sample collector; and
e) amplifying the DNA from the sperm and epithelial cells by a polymerase chain reaction (PCR);
wherein the sample is forensically analyzed by STR for DNA from the collected sperm and epithelial cells.
